(19) United States
(12) Patent Application Publication
(10) Pub. No.: US 2022/0218264 A1
(43) Pub. Date: Jul. 14, 2022

(54) METHOD TO PERFORM SPECTRAL BIOPSY OF ELECTROPHYSIOLOGICAL BRAIN FUNCTION

(71) Applicant: Washington University, St. Louis, MO (US)

(72) Inventors: Peter Brunner, St. Louis, MO (US); Eric Leuthardt, St. Louis, MO (US); Tao Xie, Shanghai (CN); Zehan Wu, Shanghai (CN); Xinjun Sheng, Shanghai (CN); Liang Chen, Shanghai (CN); Xiangyang Zhu, Shanghai (CN)

(73) Assignees: Washington University, St. Louis, MO (US); Shanghai Jiao Tong University, Shanghai (CN); Fudan University, Shanghai (CN)

(21) Appl. No.: 17/574,524

(22) Filed: Jan. 12, 2022

Related U.S. Application Data
(60) Provisional application No. 63/136,630, filed on Jan. 12, 2021.

(51) Int. Cl. A61B 5/00; A61B 5/374; G01R 23/18

(52) U.S. Cl. CPC A61B 5/4064; A61B 5/374; G01R 23/18; A61B 5/725; A61B 5/7257; A61B 5/7246

(57) ABSTRACT

Methods and systems are disclosed for analyzing interactions between low-frequency oscillations and high-frequency activity in electromagnetic brain signals such as EEG, MEG, SEEG, and ECoG signals in subjects in real-time that does not depend on the signals being contained within narrow frequency bands, sinusoidal, sustained and monolithic. The disclosed methods and systems can be applied to electromagnetic brain signals to detect brain activity alterations associated with neurological and psychiatric diseases.

### CROSS-REFERENCE TO RELATED APPLICATIONS

This application claims priority from U.S. Provisional Application Ser. No. 63/136,630 filed on Jan. 12, 2021, which is incorporated herein by reference in its entirety.

### STATEMENT REGARDING FEDERALLY SPONSORED RESEARCH OR DEVELOPMENT

This invention was made with government support under EB018783, NS108916, NS109103, and EB026439 awarded by the National Institutes of Health. The government has certain rights in the invention.

### MATERIAL INCORPORATED-BY-REFERENCE

Not applicable.

### FIELD OF THE DISCLOSURE

The present disclosure generally relates to characterizing electrophysiological brain function.

### BACKGROUND OF THE DISCLOSURE

Investigations of brain function depend on assessing the interactions between neuronal activity occurring at different temporal scales throughout the brain. While brain function has traditionally been studied in terms of neuronal responses to environmental demands, this ignores the fact that much of the brain's energy is devoted to intrinsic neuronal signaling. This intrinsic neuronal activity manifests as spontaneous fluctuations in the brain signal. The study of such fluctuations could potentially provide insight into the brain's functional organization and lead to diagnostic tools that detect intrinsic activity altered in neurological and psychiatric diseases.

Traditional approaches to determining the intrinsic neuronal activity rely on calculating the Hilbert transform of the bandpass filtered neuronal activity to extract the amplitude and phase components. For example, Hilbert-based phase-amplitude coupling (PAC) between the phase of low-frequency oscillations and the power of high-frequency activity is performed in three steps: 1) bandpass filtering of the neuronal activity into narrow bands of interest, e.g., alpha and gamma; 2) applying a Hilbert transform to obtain amplitude and phase information from each frequency band; and 3) quantifying the correlation between phase and amplitude.

Methodological limitations within the Hilbert transform to accurately estimate the phase of natural broadband signals require filtering the neuronal activity into arbitrary narrow bands. Consequently, any application of the Hilbert transform-based phase-amplitude coupling on neural activity requires filtering the signals into narrow bands of interest. Thus, Hilbert transform-based phase-amplitude coupling is based on the assumption that the neuronal activity is: (1) contained within one narrow frequency band; (2) sinusoidal; and (3) sustained and uniform. However, natural neuronal signals often violate the assumptions, creating a potential confound for the interpretation of the resulting PAC. This makes traditional approaches to determining the intrinsic neuronal activity ill-suited for use in diagnostic tools that detect intrinsic activity altered in neurological and psychiatric diseases.

### SUMMARY

In one aspect, a computer-implemented method for tracking a brain state and mapping a functional brain organization of a subject is disclosed. The method includes receiving, at a computing device, a plurality of brain activity measurements indicative of brain activity of the subject; extracting, using the computing device, a plurality of wideband low frequency (WBLF) signals from the plurality of brain activity measurements; extracting, using the computing device, a plurality of broadband gamma envelope signals from the plurality of brain activity measurements; calculating, using the computing device, a cross-correlation between the plurality of wideband low frequency (WBLF) signals and the plurality of broadband gamma envelope signals to produce at least one Tau Modulation Curve; and displaying, using the computing device, the at least one TMC, wherein the at least one TMC is indicative of the WBLF modulation of broadband gamma activity in the brain of the subject. In some aspects, the plurality of brain activity measurements comprises at least one of electroencephalographic (EEG) signals, magnetoencephalographic (MEG) signals, electrocorticographic (ECoG) signals, stereo electroencephalography (SEEG) signals, functional magnetic resonance (fMRI) signals, and functional near-infrared optical imaging (fNRI) signals. In some aspects, extracting the plurality of WBLF signals comprises applying, using the computing device, an FIR lowpass filter (<30 Hz) to the plurality of brain activity measurements. In some aspects, the method further includes normalizing, using the computing device, the plurality of WBLF signals to remove the effect of 1/f power law scaling. In some aspects, normalizing the plurality of WBLF signals includes applying, using the computing device, a Hamming window and a fast Fourier transform to the plurality of WBLF signals to obtain a WBLF amplitude spectrum and a WBLF phase spectrum; obtaining, using the computing device, a least-squares linear regression fit of the WBLF amplitude spectrum over a 1-30 Hz log-log spaced range; normalizing, using the computing device, the WBLF amplitude spectrum using the least-squares linear regression fit to obtain a normalized amplitude spectrum; and performing, using the computing device, an inverse fast Fourier transform to the normalized amplitude spectrum to obtain a plurality of normalized WBLF signals. In some aspects, extracting the plurality of broadband gamma envelope signals comprises applying, using the computing device, an FIR bandpass filter (70-170 Hz) and a Hilbert transform to the plurality of brain activity measurements. In some aspects, the method further includes calculating, using the computing device, a TMC-strength for each of the at least one TMCs, each TMC-strength indicative of a strength of the WBLF modulation of broadband gamma activity in the brain of the subject, wherein each TMC-strength comprises a signal-to-noise ratio (SNR) for each of the at least one TMCs, each SNR comprising a ratio of an average variance of each TMC and an average variance of all of the at least one TMCs. In some aspects, a TMC-strength value of at least 1 is indicative of a presence of WBLF modulation of broadband gamma activity in the brain of the subject. In some aspects, the method further includes calculating, using the computing device, a TMC-frequency for each of the at least one TMCs, each TMC-frequency indicative of a frequency of WBLF modulation of broadband gamma activity in the brain of the subject, wherein calculating the TMC-frequency comprises calculating, using the computing device, an average TMC for each of the at least one TMCs; and applying, using the computing device, a matching pursuit filter to determine a fundamental oscillation frequency of each average TMC, wherein the fundamental oscillation frequency is the TMC-frequency. In some aspects, displaying the at least one Tau Modulation Curve (TMC) further comprises displaying, using the computing device, a TMC-strength map and a TMC-frequency map, the TMC-strength and TMC-frequency maps comprising a plurality of TMC-strengths and a plurality of TMC-frequencies mapped to a corresponding plurality of brain positions at which the subset of brain activity measurements used to produce each TMC was obtained, respectively.

In another aspect, a system for tracking a brain state and mapping a functional brain organization of a subject is disclosed that includes a computing device that includes at least one processor configured to receive a plurality of brain activity measurements indicative of brain activity of the subject; extract a plurality of wideband low frequency (WBLF) signals from the plurality of brain activity measurements; extract a plurality of broadband gamma envelope signals from the plurality of brain activity measurements; calculate a cross-correlation between the plurality of wideband low frequency (WBLF) signals and the plurality of broadband gamma envelope signals to produce at least one Tau Modulation Curve (TMC); and display the at least TMC, wherein the at least one TMC is indicative of the WBLF modulation of broadband gamma activity in the brain of the subject. In some aspects, the plurality of brain activity measurements comprises at least one of electroencephalographic (EEG) signals, magnetoencephalographic (MEG) signals, electrocorticographic (ECoG) signals, stereo electroencephalography (SEEG) signals, functional magnetic resonance (fMRI) signals, and functional near-infrared optical imaging (fNRI) signals. In some aspects, the at least one processor is further configured to extract the plurality of WBLF signals by applying an FIR lowpass filter (<30 Hz) to the plurality of signals indicative of brain activity. In some aspects, the at least one processor is further configured to normalize the plurality of WBLF signals to remove the effect of 1/f power law scaling. In some aspects, the at least one processor is further configured to normalize the plurality of WBLF signals by applying a Hamming window and a fast Fourier transform to the plurality of WBLF signals to obtain a WBLF amplitude spectrum and a WBLF phase spectrum; obtaining a least-squares linear regression fit of amplitude spectrum with a 1-30 Hz log-log spaced range; normalizing the amplitude spectrum using the least-squares linear regression fit to obtain a normalized amplitude spectrum; and performing an inverse fast Fourier transform to the normalized amplitude spectrum to obtain a plurality of normalized WBLF signals. In some aspects, the at least one processor is further configured to extract the plurality of broadband gamma envelope signals by applying an FIR bandpass filter (70-170 Hz) and a Hilbert transform to the plurality of brain activity measurements. In some aspects, the at least one processor is further configured to calculate a TMC-strength for each of the at least one TMCs, wherein each TMC-strength comprises a signal-to-noise ratio (SNR) for each of the at least one TMCs, each SNR comprising a ratio of an average variance of each TMC and an average variance of all of the at least one TMCs; and a TMC-strength value of at least 1 is indicative of a presence of WBLF modulation of broadband gamma activity in the brain of the subject. In some aspects, wherein the at least one processor is further configured to calculate a TMC-frequency for each of the at least one TMCs, each TMC-frequency indicative of a frequency of WBLF modulation of broadband gamma activity in the brain of the subject, wherein calculating the TMC-frequency comprises: calculating an average TMC for each of the at least one TMCs; and applying a matching pursuit filter to determine a fundamental oscillation frequency of each average TMC, wherein the fundamental oscillation frequency is the TMC-frequency. In some aspects, the at least one processor is further configured to display at least one of a TMC-strength map and a TMC-frequency map, the TMC-strength and TMC-frequency maps comprising a plurality of TMC-strengths and a plurality of TMC-frequencies mapped to a corresponding plurality of brain positions at which the subset of brain activity measurements used to produce each TMC was obtained, respectively In some aspects, the system further includes a brain activity monitoring device operatively coupled to the computing device, the brain activity monitoring device configured to obtain the plurality of brain activity measurements, the brain activity monitoring device comprising one of an electroencephalographic system, a magnetoencephalographic (MEG) system, an electrocorticographic (ECoG) system, a stereo electroencephalography (SEEG) system, a functional magnetic resonance (fMRI) system, and a functional near-infrared optical imaging (fNRI) system.

Other aspects of the disclosure are presented in additional detail herein.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A shows brain signals (e.g., electrocorticographic, ECoG) recorded from an electrode near the central sulcus while the subject is in rest condition (left panel, see red circle in FIG. 1C for electrode location). Wideband low-frequency (WBLF, middle panel), and high-frequency signals (right panel) extracted from the ECoG signal.

FIG. 1B shows signal normalized to its 1/f power-law characteristic. The detailed normalizing procedure includes using a Hamming window followed by a fast Fourier transform (FFT) to extract the signal's amplitude and phase spectrum, a least square regression to determine a linear fit of the amplitude spectrum within the 1-30 Hz log-log spaced range, a linear fit to normalize the amplitude spectrum while keeping the phase spectrum constant, and an inverse FFT to transform the frequency-domain signal back into the time-domain to yielded our normalized-WBLF signal.

FIG. 1C shows Tau-Modulation Curve (TMC) computed as the cross-correlation between the high-frequency signal and low-frequency signal (e.g., normalized-WBLF) for 1.28 s-long observation windows that are moved in steps of 0.04 s.

FIG. 1D shows the strength of TMC presented in the form of a signal-to-noise ratio (SNR) within each task condition. SNR is calculated as the ratio between the variance across all TMC samples, and the average variance of each individual TMC sample. SNR values>1 indicate WBLF modulating high-frequency activity. In contrast, SNR values˜=1 Indicate no modulation. SNR values are used to index the strength of TMC.

FIG. 1E shows the frequency of TMC computed as the average TMC, followed by a matching pursuit filter to accurately determine the fundamental oscillation frequency of the average TMC.

FIG. 2A shows the procedure of calculating the Tau-Modulation Curve.

FIG. 2B shows the electrodes' location and the experimental paradigms.

FIG. 2C shows the brain parcellation results using strength information of TMC.

FIG. 2D shows the brain parcellation results using frequency information of TMC. To determine the frequency of TMC, we applied a matching pursuit filter to accurately extract the fundamental oscillation frequency from each cortical location's Tau-oscillation curve and Tau-modulation curve. FIG. 2D also shows the accurate fundamental frequency of the Tau-oscillation curves for different behavioral and cognitive tasks. The size of the circles represents the SNR, while the color represents the fundamental frequency of the low-frequency oscillation. The results indicate the fundamental frequency shift varies depending on the behavioral and cognitive demands.

FIG. 2E shows the brain parcellation results using shape information of TMC. The left panel shows the Tau-modulation curve of all electrodes. We hypothesized that the shape of the Tau-modulation is indicative of its underlying functional area. To test this hypothesis we applied k-means clustering to two components of a principal component analysis (PCA) of the Tau-modulation curves (middle panel). The right panel shows the brain parcellation results using shape information.

FIG. 3A shows the procedure of calculating the Tau-Modulation Curve. We calculated the cross-frequency coupling between slow-wave oscillations (0.5-4 Hz) and high-frequency activity (55-145 Hz).

FIG. 3B shows the procedure of calculating Tau-Modulation Strength.

FIG. 3C shows that the slow-wave activity exhibits two distinct stages as it travels through the entire hemisphere throughout the Propofol-induced anesthesia.

FIG. 4 is a block diagram schematically illustrating a system in accordance with one aspect of the disclosure.

FIG. 5 is a block diagram schematically illustrating a computing device in accordance with one aspect of the disclosure.

FIG. 6 is a block diagram schematically illustrating a remote or user computing device in accordance with one aspect of the disclosure.

FIG. 7 is a block diagram schematically illustrating a server system in accordance with one aspect of the disclosure.

FIG. 8A contains a series of graphs showing the strength of TMC during each of six task conditions for an electrode positioned near the central sulcus, shown illustrated as a red circle in FIG. 8D. Tasks are defined as: solving Rubik's cube (T1), tickling chin (T2), sticking out tongue (T3), pursing lips (T4), listening (T5), and resting (T6).

FIG. 8B contains a series of graphs showing the frequency of TMC measured during each of six task conditions by an electrode positioned near the central sulcus as described in FIG. 8A.

FIG. 8C contains graphs comparing power spectral densities (PSDs) of the raw signals measured during each task condition by an electrode positioned near the central sulcus as described in FIG. 8A. The upper graph compares PSDs within a 50 Hz frequency range and the lower graph compares the same PSDs within frequencies ranging from 5 Hz to 10 Hz.

FIG. 8D is a map of the electrocorticography (ECoG) grid coverage (black dots) as well as the position of an electrode near the central sulcus (red circle) used to obtain the measurements analyzed to obtain the results summarized in FIGS. 8A, 8B, 8C, 8E, 8F, and 8G.

FIG. 8E is a series of maps showing the spatial distribution of the TMC strength for each of the tasks described in FIG. 8A.

FIG. 8F is a series of maps showing the spatial distribution of the Modulation Index (MI) for each of the tasks described in FIG. 8A.

FIG. 8G is a series of graphs showing the Spearman correlation (r) of the TMC strength and MI across all electrodes for each of the tasks described in FIG. 8A.

FIG. 9 contains maps of EcoG electrode positions across the lateral and medial surfaces of the left hemispheres of two monkeys, described further in Example 2.

FIG. 10 is a flow chart illustrating a method for Tau-modulation analysis of brain activity measurements in accordance with one aspect of the disclosure.

FIG. 11 is a block diagram illustrating a system for Tau-modulation analysis of brain activity measurements in accordance with one aspect of the disclosure.

### DETAILED DESCRIPTION OF THE DISCLOSURE

I. Tau-Modulation Analysis Theory

In various aspects, Tau-Modulation analysis is used to extract the relationship between the phase of low-frequency oscillations and the amplitude of high-frequency activity in the brain. In contrast to traditional Hilbert transform-based phase-amplitude coupling-based methods, the disclosed method does not depend on neuronal activity being contained within a narrow frequency band, being sinusoidal, or being sustained. Thus, Tau-Modulation is a more robust approach to analyzing the spectral interactions within neuronal signals, paving the way for using Tau-Modulation as a spectral biopsy of human brain function. Thus, Tau-Modulation as a spectral biopsy method serves as the methodological basis for diagnostic methods that characterize pathologies directly from the brain's activity.

Recent studies suggest that phase-amplitude coupling (PAC) between the phase of low-frequency oscillations and the power of high-frequency activity plays a functional role in neuronal computation, communication, and learning (see generally Fries, P. Neuron 88:220-235, 2015). This is further supported by studies that showed that high-frequency brain activity reflects local cortical processing, while low-frequency brain oscillations are dynamically entrained across brain regions by both external sensory input and internal cognitive events (see generally Canolty, R. T. et al. Science 313:1626-1628, 2006). PAC might thus serve as a mechanism to transfer information from large-scale brain networks operating at behavioral timescales to the fast, local cortical processing required for effective computation and synaptic modification, thus integrating functional systems across multiple spatiotemporal scales (see generally Canolty, R. T. & Knight, R. T. Trends Cogn. Sci. 14:506-515, 2010).

Conventional methods that compute PAC rely on calculating the Hilbert transform of the bandpass filtered neuronal activity to extract the amplitude and phase components. Thus, PAC is typically performed in three steps: (1) bandpass filtering of the neuronal activity into narrow bands of interest, e.g., alpha and gamma; (2) applying a Hilbert transform to obtain amplitude and phase information from each frequency band; and (3) quantifying the correlation between phase and amplitude. Methodological limitations within the Hilbert transform to accurately estimate the phase of natural broadband signals require filtering the neuronal activity into arbitrary narrow bands (see e.g., Cohen, M. X. eLife 6:e21792, 2017). Consequently, any application of the Hilbert transform-based phase-amplitude coupling on neural activity requires filtering the signals into narrow bands of interest. Thus, Hilbert transform-based phase-amplitude coupling is based on the assumption that the neuronal activity is: (1) contained within one narrow frequency band; (2) sinusoidal; and (3) sustained and monolithic. However, natural neuronal signals often violate the assumptions, as explained in the following paragraphs.

Assumption 1: Neuronal networks within the human brain demonstrate several oscillatory bands covering the range from approximately 0.05 to 500 Hz. Each frequency band is associated with a specific brain state, and these frequency bands compete and interact with each other. Studies have found distinct oscillatory bands of intrinsic neuronal networks. For example, Hipp et al. found that spontaneous oscillatory neuronal activity within the brain exhibits a frequency-specific spatial correlation structure. They found theta frequency (4-6 Hz) in the medial temporal lobe, alpha to beta frequency (8-23 Hz) in the lateral parietal areas, and higher frequencies (32-45 Hz) in the sensorimotor areas (see e.g., Hipp, J. F., et al. Nat. Neurosci. 15:884-890, 2012).

Assumption 2: There is increasing evidence that neural oscillations are commonly non-sinusoidal. The specific shape of the waveform reflects the physiological aspects of the underlying cortical area. The nomenclature of the neuronal oscillations is derived from the oscillation's shape. For example, the shape of sensorimotor mu “rhythm” resembles the Greek character “mu”, and the beta oscillation within the motor cortex manifests a sawtooth shape. These non-sinusoidal shapes are also known to represent crucial physiological features related to neuronal computation, communication, and cognition (see e.g., Cole, S. R. & Voytek, B. Trends Cogn. Sci. 21:137-149, 2017).

Assumption 3: Traditional oscillations exhibit repeated cycles of neuronal activity with a reliable periodicity. This characteristic is most prominently exhibited within alpha oscillations recorded from the occipital cortex during eyes-closed conditions, within oscillations during sleep, and within theta oscillations recorded from the hippocampus. However, recent evidence shows that in many instances, brain signals commonly attributed to a frequency-defined class of brain oscillation do not represent sustained oscillatory activation, but rather brief bursts of activity that are repeated intermittently. This intermittency has direct implications on the interpretation of oscillations (see e.g., Jones, S. R. Current Opinion in Neurobiology 40:72-80, 2016).

In summary, the neuronal activity often does not meet the three assumptions of the Hilbert transform, creating a potential confound for the interpretation of PAC results. As disclosed herein, we present a new method that overcomes this limitation by requiring fewer assumptions to be met, and that enables diagnostic tools that detect brain activity altered in neurological and psychiatric diseases.

II. Tau-Modulation Analysis Method

In various aspects, a Tau-Modulation analysis method is disclosed that quantifies PAC without imposing any underlying assumptions about the underlying neuronal oscillations as described above. The disclosed Tau-Modulation analysis method overcomes at least a portion of the limitations of the existing method that make use of the Hilbert-transform and its associated assumptions described above.

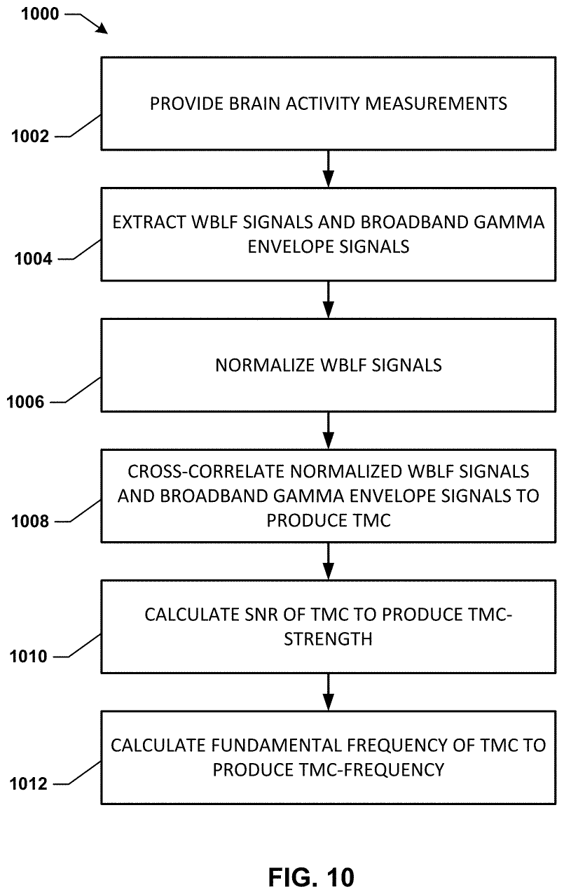

In various aspects, a Tau-Modulation analysis method is illustrated in the form of a flow chart as shown in FIG. 10. The disclosed method 1000 includes providing brain activity measurements at 1002. Any suitable measurements of brain activity may be provided without limitation. Non-limiting examples of suitable brain activity measurements include electromagnetic (EM) signals indicative of brain activity or other physiological systems such as heart activity or muscle activity. Non-limiting examples of suitable electromagnetic (EM) signals indicative of brain activity include electroencephalographic (EEG) signals, magnetoencephalographic (MEG) signals, electrocorticographic (ECoG) signals, electroencephalography (SEEG) signals, functional magnetic resonance (fMRI) signals, functional near-infrared optical imaging (fNRI) signals, functional optical coherence tomography (fOCT) signals, and any other suitable signals indicative of brain activity.

In various aspects, the brain activity measurements provided at 1002 may be obtained from a subject under a variety of conditions, activities, and states without limitation. In some aspects, the brain activity measurements may be obtained from a subject in a variety of conditions including, but not limited to, a resting condition, an active condition of performing or attending to a task, while receiving a stimulus, and any combination thereof. In one aspect, the brain activity measurements may be obtained while a subject is communicating truthfully (i.e. speaking the truth) or communicating deceitfully (i.e. telling a lie). In other aspects, the brain activity measurements may be obtained from a subject with brain activity characterized as normal or abnormal brain activity. Non-limiting examples of abnormal brain activity include brain activity altered in neurological and psychiatric diseases.

Referring again to FIG. 10, method 1000 further includes extracting at 1004 two signal subsets characterized by different frequency ranges from the brain activity measurements provided at 1002. In various aspects, the two extracted signal subsets correspond to low-frequency brain activity signals and high-frequency brain activity signals, respectively. In some aspects, the signal subset corresponding to low-frequency brain activity signals comprises wideband low-frequency (WBLF) signals, defined herein as a subset of brain activity measurements with frequencies of less than about 30 Hz. In some aspects, the signal subset corresponding to high-frequency brain activity signals comprises a broadband gamma envelope, defined herein as a subset of brain activity measurements with frequencies ranging from about 70 Hz to about 170 Hz.

In various aspects, the wideband low-frequency (WBLF) signals may be extracted from the brain activity measurements using any existing means without limitation. In one exemplary aspect, the wideband low-frequency (WBLF) signals may be extracted by applying a finite impulse response (FIR) lowpass filter (<30 Hz) to the brain activity measurements. Without being limited to any particular theory, the wideband characteristics of this filter preserve the shape and amplitude of the neuronal oscillations captured in the brain activity measurements.

In various aspects, the broadband gamma envelope signals may be extracted from the brain activity measurements using any existing means without limitation. In one exemplary aspect, the wideband low-frequency (WBLF) signals may be extracted by applying an FIR bandpass filter to the brain activity measurements followed by a Hilbert transform to extract signals with frequencies of 70-170 Hz.

In various aspects, the brain activity measurements may be pre-processed prior to further analysis using any suitable data analysis and/or signal processing methods known in the art without limitation. In some aspects, pre-processing may be used to remove a variety of noise and artifacts from the brain activity measurements. Non-limiting examples of noise and artifacts that may be removed from the brain activity measurements during pre-processing include: signals from non-functional electrodes, slow drifts in the signal, line noise and or noise harmonics, and any other appropriate noise or artifact of the brain activity signals. By way of non-limiting example, a high-pass finite impulse response (FIR) filter at 0.5 Hz may be used to remove slow drifts. By way of another non-limiting example, a common average reference spatial filter may be used to remove common noise across all channels. In another additional non-limiting example, one or more notch FIR filters at one or more frequency ranges may be used to remove line noise and its first harmonic. Non-limiting examples of suitable frequency ranges for the notch FIR filters include 58-62 Hz and 118-122 Hz.

Referring again to FIG. 10, the WBLF signals extracted from the brain activity measurements at 1004 are normalized at 1006. Similar to any other naturally occurring signal, the power density of neuronal activity exhibits an inverse relationship between signal power and frequency, i.e., 1/f power-law scaling. Because all oscillations within the wideband low-frequency signal are of interest in analyzing phase-amplitude coupling (PAC) using the disclosed method 1000, the WBLF signals are normalized at 1006 to remove this 1/f characteristic to produce a normalized-WBLF signal.

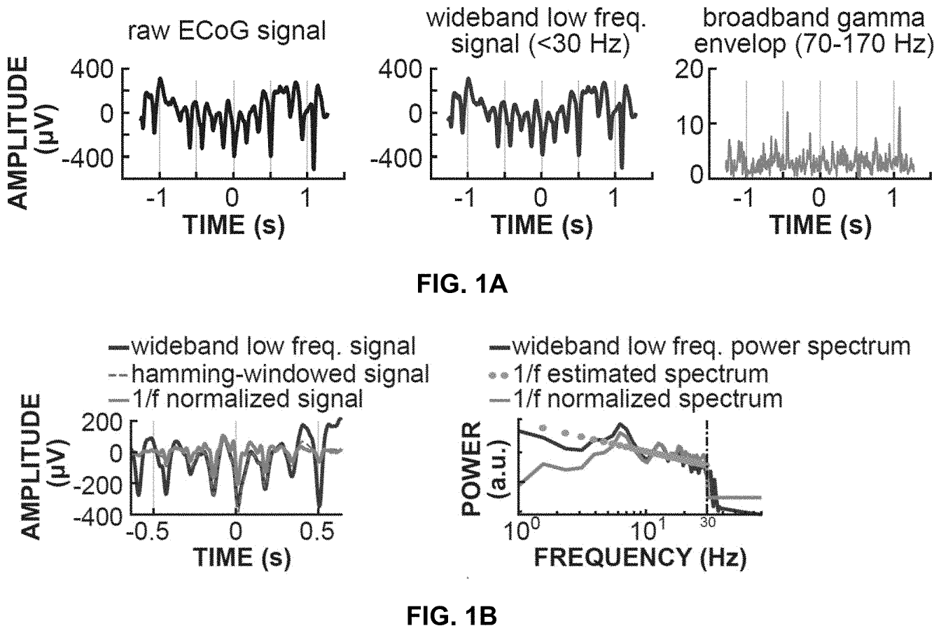

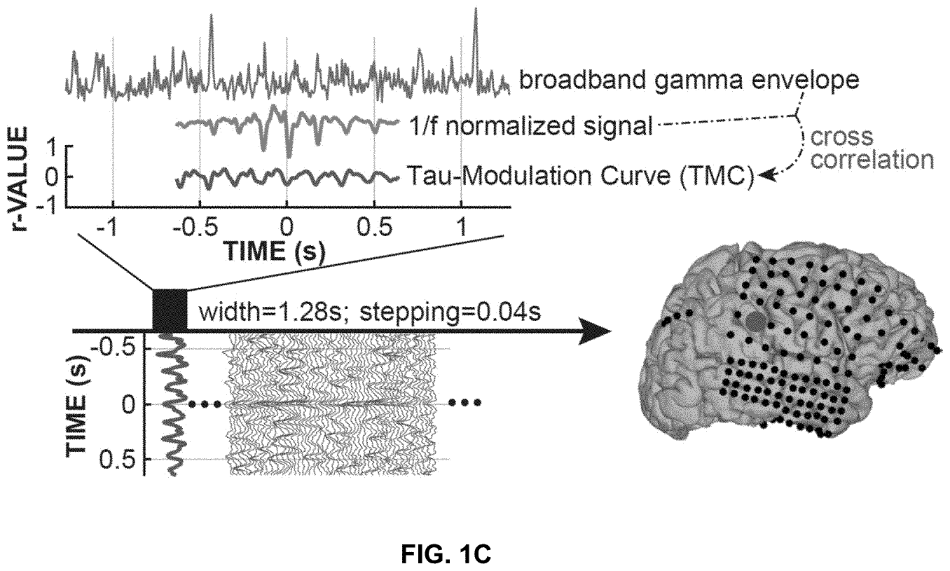

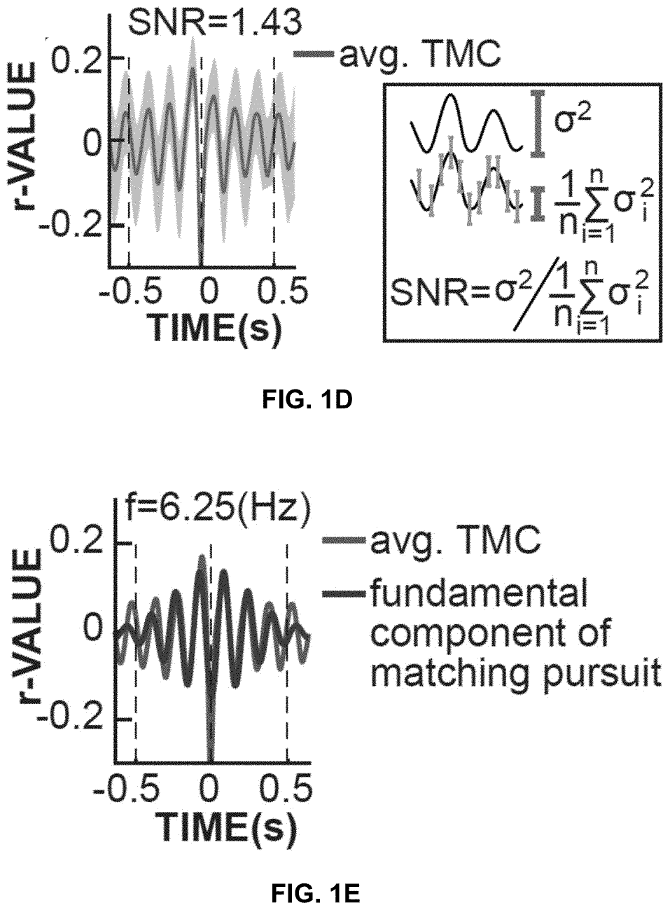

In various aspects, the WBLF signals may be normalized using any suitable method known in the art without limitation. By way of non-limiting example, a Hamming window followed by a fast Fourier transform (FFT) may be used to extract the amplitude and phase spectrum of the WBLF signals. A least square regression may be performed to determine a linear fit of the amplitude spectrum within the 1-30 Hz log-log spaced range. Using this linear fit, the amplitude may be normalized while keeping the phase spectrum constant. This normalized amplitude spectrum may be transformed back into a time-domain using an inverse FFT to produce the normalized-WBLF signal, as illustrated in FIG. 1B.

Referring again to FIG. 1, the method may further include calculating a cross-correlation between the normalized-WBLF and the high-frequency gamma activity envelope to yield the Tau-Modulation Curve (TMC) at 1008. Without being limited to any particular theory, the TMC encodes the strength and frequency of the WBLF coupling with the high-frequency activity and serves as the spectral biopsy of electrophysiological brain function.

Referring again to FIG. 10, method 1000 may further include analyzing the TMC to assess the strength and frequency of the TMC, which are indicative of the strength and frequency of phase-amplitude coupling, as described above. In one aspect, method 1000 further includes calculating the signal-to-noise ratio of the TMC to produce a TMC-strength at 1010. SNR, as used herein, is defined as the ratio between the variance across all TMC samples, and the average variance of each individual TMC sample. Without being limited to any particular theory, each SNR value serves as an indication of the strength of phase-amplitude coupling (PAC) between WBLF and broadband gamma activity for a particular condition, state, or activity of the subject from which the brain activity measurements were obtained. In some aspects, SNR>1 indicates that WBLF is modulating broadband gamma activity via PAC and SNR≈1 indicates no modulation.

Referring again to FIG. 10, the TMC may be further analyzed to produce a TMC-frequency at 1012. In various aspects, the TMC-frequency may be a fundamental frequency of the TMC produced using any suitable method known in the art without limitation. In one aspect, the TMC-frequency may be produced by applying a matching pursuit filter to a TMC (or a mean of multiple TMCs determined for multiple instances of the same state/condition/activity of the subject) to determine a fundamental oscillation frequency indicative of the TMC-frequency.

The ability to have real-time measures of brain regions that are functional or state-specific has at least several high-impact applications. Potential applications of Tau-Modulation Analysis of brain activity include:

Intraoperative brain mapping. Using the spectral biopsy methodology could enable a neurosurgeon to determine the function of a particular region of the brain merely by recording a few minutes of its intrinsic signals. This would be deeply important for surgical resections of brain tumors and epilepsy foci. Additionally, this could provide critical information for the optimal positioning of electrodes in the course of implantation of brain-computer interfaces or neuromodulation systems.

Monitoring of changing brain state. There are several existing technologies (some commercially available) that attempt to monitor changes in brain state, most notably, anesthesia monitoring. These existing technologies often lack sufficient accuracy to enable meaningful interpretation and clinical use. The proposed Tau-modulation approach would likely be a much more effective anesthesia monitoring system.

Network interactions are associated with specific frequency rhythms. Additionally, these rhythmically encoded networks can become distorted in various psychiatric and neurologic diseases. The method could be useful in potentially measuring changes in mood and other neurologic conditions.

Although the tau-modulation analysis methods are disclosed in terms of brain activity measurements herein, it is to be noted that the disclosed tau-modulation analysis methods may be used to evaluate the relationship and interactions between wide-band low-frequency signals and high-frequency signals of any biological, mechanical, electrical, or other system without limitation.

As used herein, the term “brain function” refers broadly to the functional organization of the brain that gives rise to neuronal activity.

As used herein, the term “neuronal activity” refers broadly to the brain's activity.

As used herein, the term “intrinsic neuronal activity” refers broadly to neuronal activity produced spontaneously by the brain and not as responses to stimulation or immediate reactions to the environment.

As used herein, the term “extrinsic neuronal activity” refers broadly to neuronal activity produced in responses to stimulation or immediate reactions to the environment.

As used herein, the term “local neuronal excitability” refers broadly to the strength of the response of neurons to a given stimulation, reflects neuron reactivity and response specificity, and is, therefore, a fundamental aspect of human brain function.

As used herein, the term “brain signal” refers broadly to any signal reflecting the electromagnetic (EM) activity of the brain, without limitation electroencephalographic activity (EEG), electrocorticographic (ECoG) activity, stereo electroencephalographic (SEEG) activity, or magnetoencephalographic (MEG) activity.

As used herein, the term “neurological diseases” refers broadly to any condition that involves malfunction of or damage to the nervous system including the brain, spinal cord, and nerves.

As used herein, the term “psychiatric diseases” refers broadly to any condition that involves a disturbed behavior and emotional state.

As used herein, the term “diagnostic tool” refers broadly to any system or method that assists in the diagnosis of neurological and psychiatric disease.

As used herein, the term “low-frequency oscillation” refers broadly to any brain signal oscillation within a defined spectral band.

As used herein, the term “high-frequency activity” refers broadly to any brain signal with a frequency higher than that of a low-frequency oscillation.

As used herein, the term “wideband” refers broadly to any brain signal that is not filtered into a fraction of its defined spectral bandwidth.

As used herein, the term “1/f power-law characteristic” refers broadly to the inverse relationship between frequency and spectral power in brain signals.

As used herein, the term “bandpass filtered neuronal activity” refers broadly to any brain signal that is not filtered into a fraction of its spectral bandwidth.

As used herein, the term “amplitude” refers broadly to the magnitude of the brain signal.

As used herein the term “phase” refers broadly to the relationship between the position of the amplitude crests and troughs of a low-frequency oscillation.

As used herein, the term “phase-amplitude coupling” refers broadly to the relationship between the phase and the amplitude between two frequency components of brain signals, including the low-frequency oscillation and high-frequency activity.

As used herein, the term “task” refers broadly to a mental or physical activity performed by a subject possibly in response to a specific instruction or stimulus.

As used herein, the term “stimulus” refers broadly to an external event that can be sensed through touch, sight, hearing, smell, taste, spatial orientation, proprioception (body position), and nociception (pain).

As used herein, the term “real-time” refers to a speed of computer analysis of signals of complex systems as described herein, where the speed is sufficient to accurately provide to an observer (e.g., a scientist or physician) an indicator of system events as they are occurring.

As used herein, the term “Tau-Modulation Curve (TMC)” refers broadly to the cross-correlation between the low-frequency signal and the high-frequency signal.

As used herein, the term “Tau-Modulation Strength” refers broadly to the strength of phase-amplitude coupling between the low-frequency and high-frequency, which can be indexed by the signal-to-noise ratio (SNR).

As used herein, the term “signal-to-noise ratio (SNR)” refers broadly to the ratio of signal power to the noise power.

As used herein, the term “spectral biopsy” refers broadly to the characterization of interactions between frequency bands within brain signals.

Methods and systems consistent with the present disclosure are generally directed to detecting phase-amplitude coupling with fewer assumptions to be met.

III. Computing Systems and Devices

In various aspects, the tau-modulation analysis methods disclosed herein may be implemented using at least one or more computing systems and/or methods as described below.

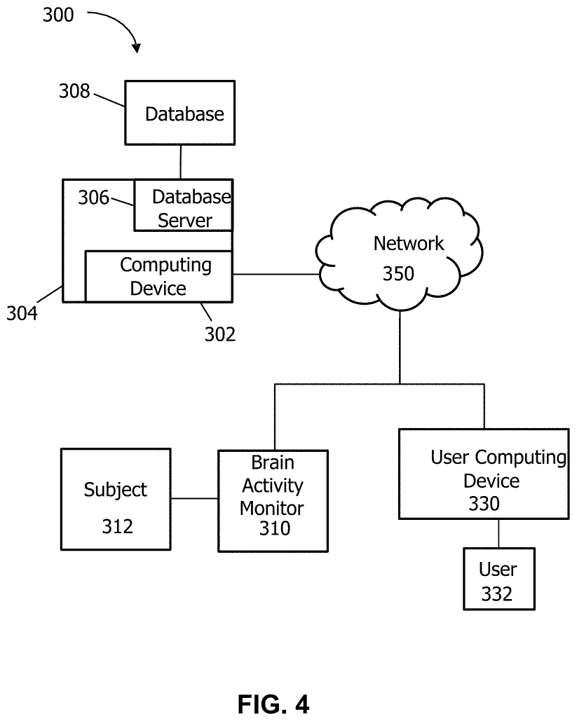

FIG. 4 depicts a simplified block diagram of a computing device 300 for implementing the methods described herein. As illustrated in FIG. 4, the computing device 300 may be configured to implement at least a portion of the tasks associated with the disclosed method. The computer system 300 may include a computing device 302. In one aspect, the computing device 302 is part of a server system 304, which also includes a database server 306. The computing device 302 is in communication with database 308 through the database server 306. The computing device 302 is communicably coupled to a brain activity monitor 310 and a user computing device 330 through a network 350.

In various aspects, the brain activity monitor 310 is configured to obtain a plurality of brain activity measurements or signals indicative of the brain activity of a subject 312. In some aspects, the brain activity monitor 310 may include at least one sensor (not illustrated) attached to the subject or implanted within the subject. The at least one sensor is configured to detect a plurality of signals including, but not limited to, EM signals that are indicative of brain activity.

Network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

In various aspects, the brain activity monitor 310 includes any system, scanner, system, or device for monitoring electromagnetic signals or other signals indicative of brain activity in a subject. In some aspects, the brain activity monitor 310 may include sensors and associated circuitry and hardware for obtaining and storing a plurality of signals indicative of brain activity. In various aspects, the brain activity monitor 310 includes, but is not limited to, at least one of a plurality of electroencephalographic (EEG) recording electrodes, a plurality of electrocorticographic (ECoG) recording electrodes, a plurality of stereo electroencephalography (SEEG) recording electrodes, an fMRI scanning device, and a functional near-infrared optical imaging or spectroscopy device. Non-limiting examples of signals indicative of brain activity in the subject include electromagnetic (EM) signals, electroencephalographic (EEG) signals, magnetoencephalographic (MEG) signals, electrocorticographic (ECoG) signals, stereo electroencephalography (SEEG) signals, functional magnetic resonance (fMRI) signals, and functional near-infrared optical imaging (fNRI) signals.

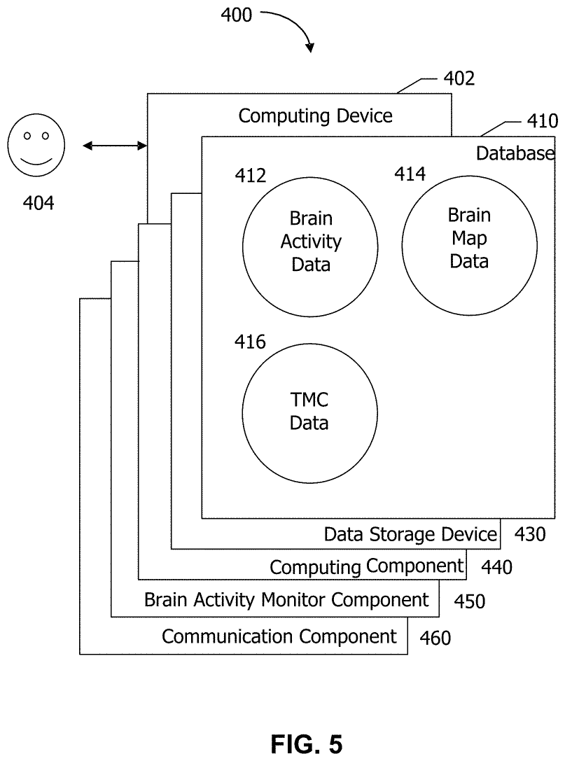

In other aspects, the computing device 302 is configured to perform a plurality of tasks associated with the disclosed method. FIG. 5 depicts a component configuration 400 of computing device 402, which includes database 410 along with other related computing components. In some aspects, computing device 402 is similar to computing device 302 (shown in FIG. 4). A user 404 may access components of computing device 402. In some aspects, database 410 is similar to database 308 (shown in FIG. 4).

In one aspect, database 410 includes various data including, but not limited to, brain activity data 412, brain map data 414, and TMC data 416. Non-limiting examples of suitable brain activity data 412 include any measurements obtained from the subject using any brain activity monitor as described herein. Non-limiting examples of suitable brain map data 414 include any data indicative of brain structure or morphology for use in mapping the TMCs or related parameters to the brain of the subject including, but not limited to, MR or CT brain images. Non-limiting examples of suitable TMC data 416 include any values of parameters defining the algorithms of the disclosed tau-modulation analysis method, such as any of the parameters associated with the continuous calculation, visualization, and tracking of the tau-modulation curves (TMC) biopsy as described above, as well as TMC-derived quantities including, but not limited to TMC-strength and TMC-frequency.

Computing device 402 also includes a number of components that perform specific tasks. In the exemplary aspect illustrated in FIG. 4, computing device 402 includes a data storage device 430, computing component 440, brain activity monitor component 450, communication component 460, and tau-modulation component 470. Data storage device 430 is configured to store data received or generated by computing device 402, such as any of the data stored in database 410 or any outputs of processes implemented by any component of the computing device 402. Computing component 440 is configured to perform the tasks associated with the tau-modulation analysis method described herein in various aspects. Brain activity monitor component 450 is configured to operate a brain activity monitor device to obtain the brain activity measurements that are provided to the tau-modulation analysis method as described herein.

Communication component 460 is configured to enable communications between computing device 402 and other devices (e.g. user computing device 330 and brain activity monitor 310, shown in FIG. 4) over a network, such as network 350 (shown in FIG. 4), or a plurality of network connections using predefined network protocols such as TCP/IP (Transmission Control Protocol/Internet Protocol). In some aspects, communication component 460 may be further configured to communicate with the subject to facilitate the recording of brain activity measurements by the brain activity monitor by providing cues to the subject to initiate a desired behavioral or cognitive activity.

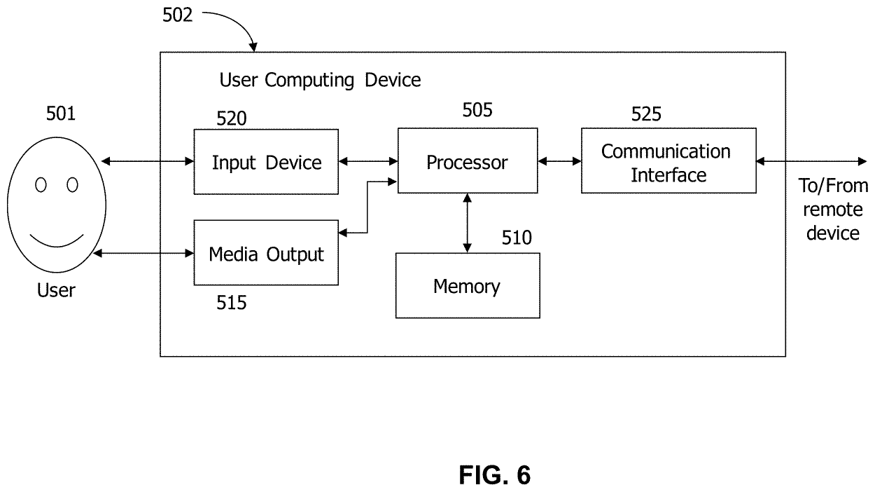

FIG. 6 depicts a configuration of a remote or user computing device 502, such as user computing device 330 (shown in FIG. 4). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively couplable to an output device such as a display device (e.g., a liquid crystal display (LCD), an organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively couplable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data networks (e.g., Worldwide Interoperability for Microwave Access (WI MAX)).

Stored in memory area 510 are, for example, computer-readable instructions for providing a user interface to user 501 via media output component 515 and, optionally, receiving and processing input from input device 520. A user interface may include, among other possibilities, a web browser, and a client application. Web browsers enable users 501 to display and interact with media and other information typically embedded on a web page or a website from a web server. A client application allows users 501 to interact with a server application associated with, for example, a vendor or business.

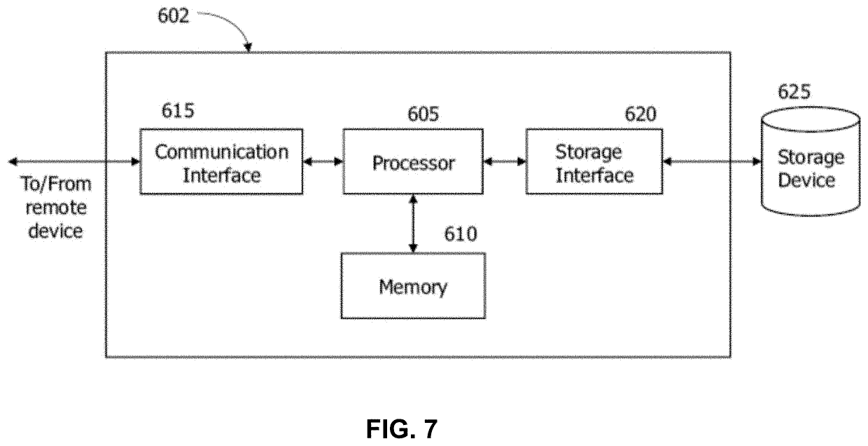

FIG. 7 illustrates an example configuration of a server system 602. Server system 602 may include, but is not limited to, database server 306 and computing device 302 (both shown in FIG. 4). In some aspects, server system 602 is similar to server system 304 (shown in FIG. 4). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as a user computing device 330 (shown in FIG. 4) or another server system 602. For example, communication interface 615 may receive requests from the user computing device 330 via a network 350 (shown in FIG. 4).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 6) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to: images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive data input, utilize a decision-making model to generate an ML output based on the data input, receive a reward signal based on the reward signal definition and the ML output, and alter the decision-making model to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application-specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for the storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Wash.). In yet another aspect, the system is run on a mainframe environment and a UNIX® server environment (UNIX is a registered trademark of X/Open Company Limited located in Reading, Berkshire, United Kingdom). The application is flexible and designed to run in various environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independent and separate from other components and processes described herein. Each component and process can also be used in combination with other assembly packages and processes. The present aspects may enhance the functionality and functioning of computers and/or computer systems.

IV. Tau-Modulation Analysis Systems

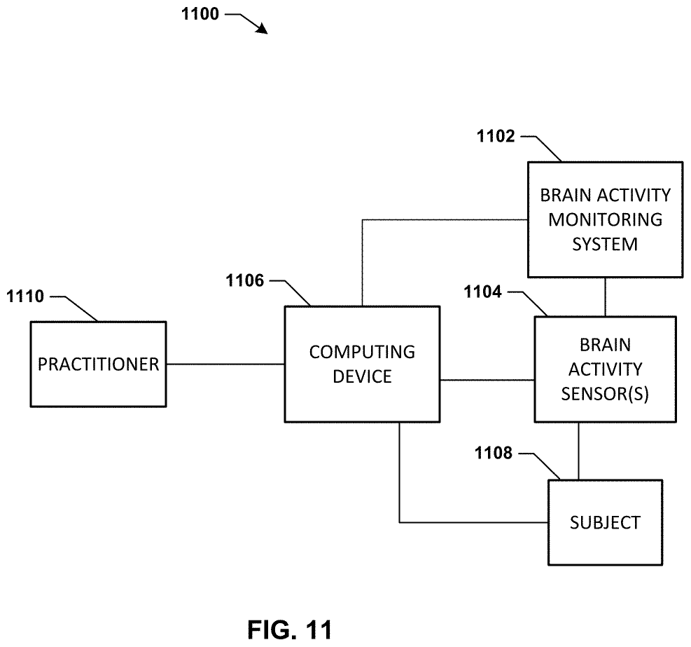

In various aspects, the tau-modulation analysis methods disclosed herein may be incorporated into a tau-modulation analysis system 1100, as illustrated in FIG. 11. In various aspects, the system 1100 is configured to continuously monitor and analyze the phase-amplitude coupling between WBLF and broadband gamma activity in real-time. In some aspects, the system 1100 is further configured to display data produced using the tau-modulation analysis methods, including, but not limited to, TMC-strengths or TMC-frequencies mapped to brain structures as disclosed herein, to a practitioner 1110. In various aspects, system 1110 includes a computing device 1106 operatively coupled to a brain activity monitoring system 1102 configured to continuously obtain brain activity measurements as disclosed herein. In some aspects, the brain activity monitoring system 1102 may obtain measurements using non-contacting sensors dedicated sensors, as is the case with fMRI scanners, for example. In other aspects, the brain activity monitoring system obtains measurements using brain activity sensors 1104 operatively coupled to the brain activity monitoring system 1102. In these other aspects, the brain activity sensors may be electrodes attached to or implanted into the subject 1104 including, but not limited to, EEG electrodes, ECoG electrodes, and the like.

In various aspects, the computing device 1106 is configured to receive brain activity measurements from the brain activity monitoring system 1102 and/or the brain activity sensors and to analyze the brain activity measurements using the tau-modulation analysis method as described herein. In some aspects, the computing device 1106 is further configured to provide cues to the subject 1108 to facilitate the measurement of brain activity and the analysis of brain activity signals. By way of non-limiting example, the computing device 1106 may display cues used to initiate and terminate cognitive or behavioral tasks performed by the subject. In some aspects, the computing device 1106 may include monitors, speakers, or other communication devices to facilitate communication with the subject 1108. In other aspects, dedicated or modular monitors, speakers, or other external communication devices are operatively coupled to the computing device to facilitate communicating with the subject 1108.

In various other aspects, the computing device 1106 is further configured to display the results of the tau-modulation analysis method to a practitioner 1110 for use in a variety of applications including, but not limited to, surgical planning, monitoring of the subject's cognitive state, or any other suitable clinical, perioperative, clinical, or other suitable applications without limitation. By way of non-limiting example, the computing device 1106 may display brain maps of TMC-strength or TMC-frequency to a practitioner in real-time as a subject performs a variety of tasks. In some aspects, the computing device 1106 may include monitors, speakers, or other communication devices to facilitate communication with the practitioner 1110. In other aspects, dedicated or modular monitors, speakers, or other external communication devices are operatively coupled to the computing device to facilitate communicating with the practitioner 1110.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

### EXAMPLES

The three examples provided below demonstrate various aspects of the disclosure: functional brain parcellation (Example 1), tracking of brain state (Example 2), and tracking of mood and attention (Example 3). Appendices I and II, the contents of which are incorporated herein in their entirety, provide additional details of the experiments described in Examples 1, 2, and 3 below.

### Example 1: Functional Brain Parcellation

Brain function has traditionally been studied in terms of neuronal responses to extrinsic stimuli/tasks. However, this approach ignores the fact that much of the brain's energy is devoted to intrinsic neuronal activities. Recent studies indicate that intrinsic neuronal activity can potentially provide insight into the brain's functional organization and has been employed for preoperative functional brain mapping in patients undergoing neurosurgery. We aim to obtain an intrinsic functional parcellation of the brain using a phase-amplitude coupling method. The phase-amplitude coupling can provide a plausible physiological mechanism for both task and intrinsic functional connectivity. The low-frequency phase reflects local neuronal excitability, while high-frequency amplitude (i.e., high-frequency) reflects a general population synaptic activity. Contemporary methods that calculate phase-amplitude coupling all require a set of assumptions to be met by the intrinsic neuronal activity. This is problematic as the neuronal activity often does not meet these requirements, creating a potential confound for the interpretation of the phase-amplitude coupling results. We will present a new method that overcomes this limitation and requires fewer assumptions.

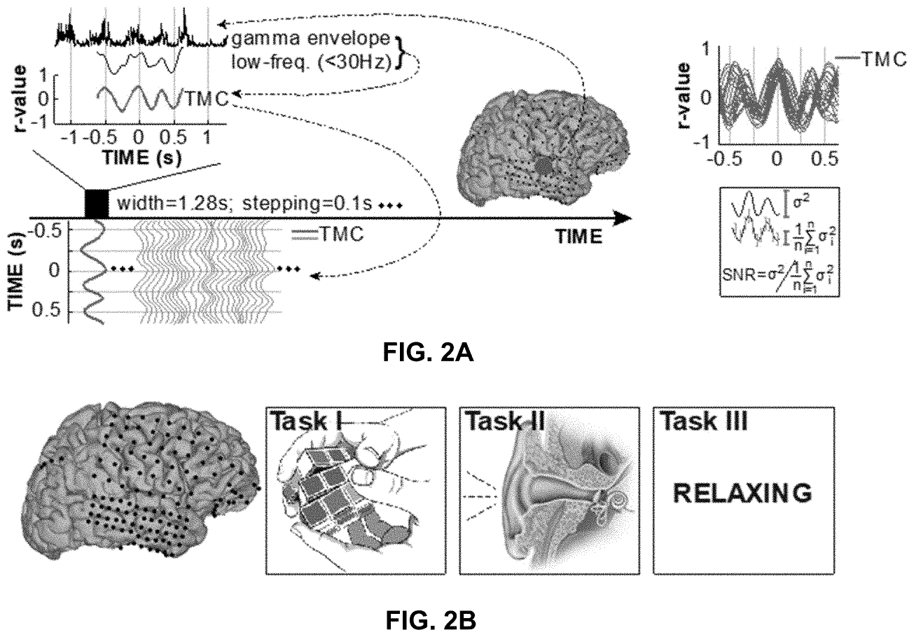

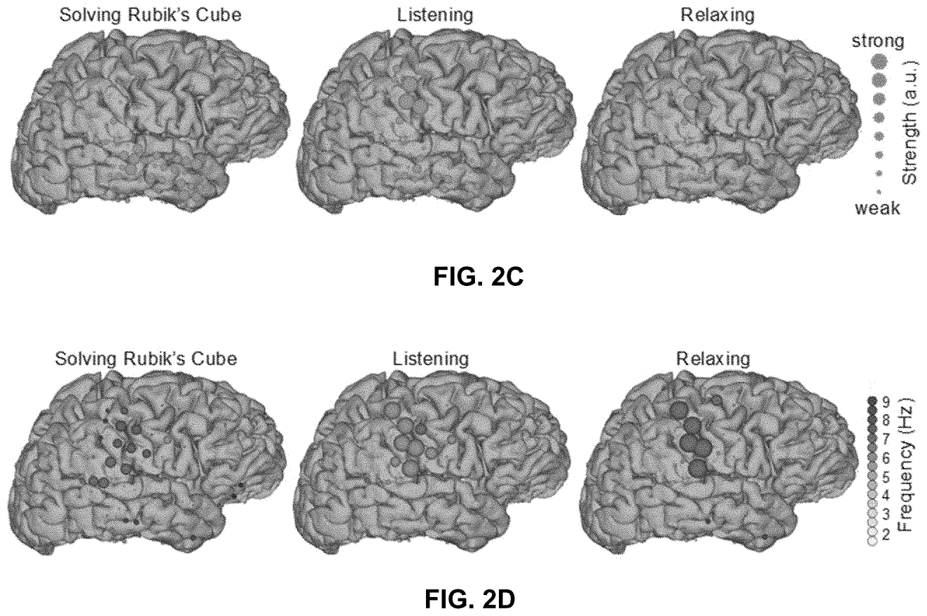

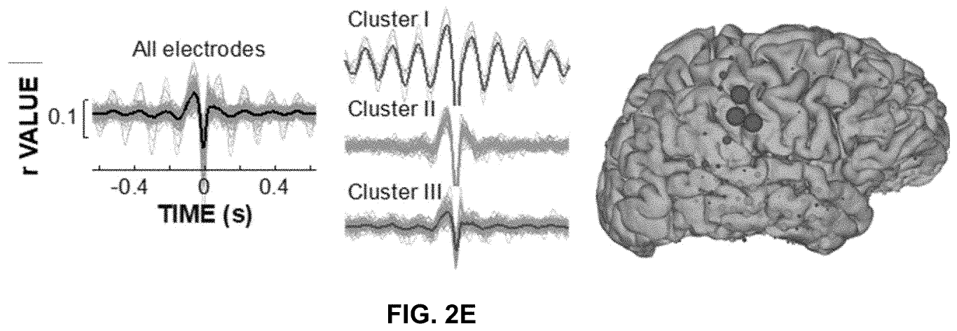

One human subject who underwent electrical cortical stimulation mapping prior to surgical resection of epileptogenic focus was involved in this study. The Institutional Review Board of Albany Medical College (Albany, N.Y.) approved the study, and the subject provided informed written consent. The Subject was implanted with several subdural ECoG grids (FIG. 2B). Electrodes had an exposed 3 mm diameter and were spaced 6-10 mm. ECoG grids were surgically implanted for around one week according to clinical indications. ECoG grid coverage and implant duration were dictated solely by the clinical requirements without considering the research study. After ECoG electrode grids implantation, we generated patient-specific brain models based on pre-operative T1-weighted structural magnetic resonance images (MRI) using the FreeSurfer software. Next, we co-registered MRI and post-operative recorded computed tomography (CT) scans to localize electrode locations on patient-specific brain models. Subsequently, we used the NeuralAct toolbox and MATLAB 2015a (The Math Works, Inc., Natick, Mass., United States) to generate brain plots. The subject performed three different tasks (FIG. 2B): manipulating a Rubik's cube, listening to a story, and relaxing.

Recent studies demonstrated that the strength of phase-amplitude coupling varies across brain areas and across behavioral and cognitive tasks. This variability might serve as a mechanism to transfer information between large-scale brain networks and local cortical functional areas. FIG. 2C shows the strength of the Tau-modulation curves that summarize how different behavioral and cognitive tasks engage different cortical networks. When the patient is “Solving Rubik's Cube”, the temporal lobe exhibits higher modulation strength than the regions surrounding the central sulcus. However, when the patient is “Listening”, the modulation strength reverses, i.e., the regions around the central sulcus exhibits higher modulation strength than the temporal lobe. Results indicate that this modulation strength information might be a useful index to identify task-related neuronal networks. FIG. 2D shows the brain parcellation results using frequency information of TMC. To determine the frequency of TMC, we applied a matching pursuit filter to accurately extract the fundamental oscillation frequency from each cortical location's Tau-oscillation curve and Tau-modulation curve. FIG. 2D shows the accurate fundamental frequency of the Tau-oscillation curves for different behavioral and cognitive tasks. The size of the circles represents the SNR, while the color represents the fundamental frequency of the low-frequency oscillation. The results indicate the fundamental frequency shift varies depending on the behavioral and cognitive demands. FIG. 2E shows the brain parcellation results using shape information of TMC. The left panel shows the Tau-modulation curve of all electrodes. We hypothesized that the shape of the Tau-modulation is indicative of its underlying functional area. To test this hypothesis we applied k-means clustering to two components of a principal component analysis (PCA) of the Tau-modulation curves (middle panel). The right panel shows the brain parcellation results using shape information.

### Example 2: Tracking Brain State

To characterize the spatiotemporal dynamics with which slow waves propagate across the cortex, the following experiments were conducted.

Slow-wave activity throughout the transition period between wakefulness and loss of consciousness (LOC) was analyzed as described below.

Slow waves are large-scale electrophysiological signals observed throughout the brain during the loss of consciousness (LOC) in the course of anesthesia, non-REM sleep, and coma. These waves arise from the synchronization of slow oscillations in the membrane potentials of millions of neurons and have been suggested as the default emergent activity of the underlying networks. The characteristics of slow-wave activity can be shaped by variations of physiological parameters and thus provide information about the underlying neural network. Studies suggest that slow-wave activity travels across the cortex. However, these studies were limited to single oscillation cycles, i.e., the transition from the downstate to the upstate, and did not consider the entire oscillation period. Consequently, the spatiotemporal dynamics with which slow waves propagate across the cortex are not fully understood to date.

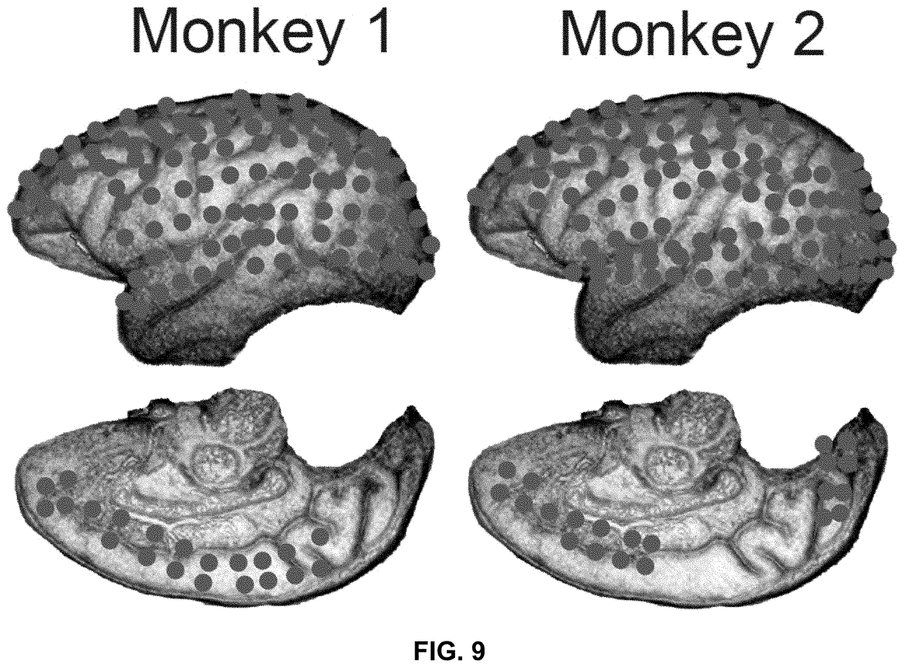

Two monkeys were implanted with Electrocorticographic (ECoG) electrodes over the lateral and medial surface of their left hemispheres as illustrated in FIG. 9. Each of the monkeys was administered propofol to induce anesthesia on two separate days, and electrocorticographic (ECoG) signals were obtained from the lateral surface over the course of propofol-induced LOC.

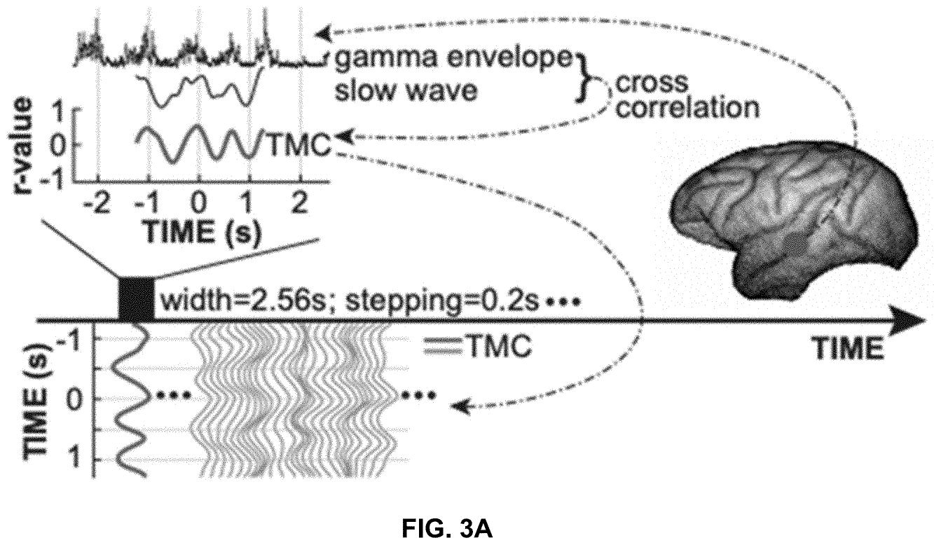

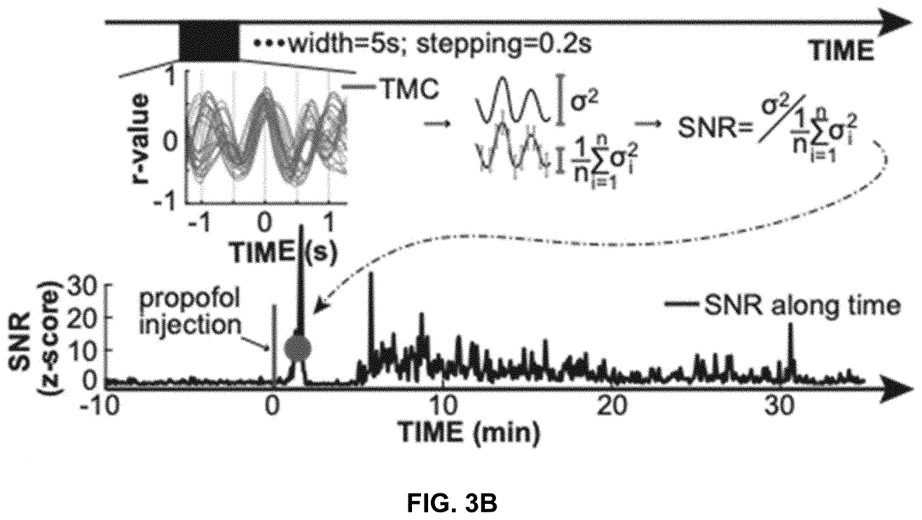

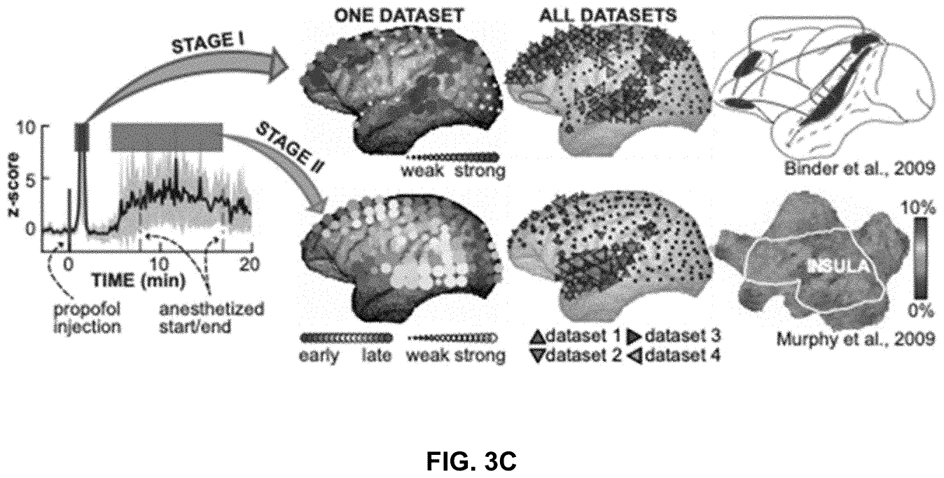

Subsequent analysis of the ECoG signals was conducted to determine the spatiotemporal dynamics of the slow-wave activity throughout the different stages of the Propofol-induced LOC. To extract the slow wave activity, the cross-frequency coupling between slow wave oscillations (0.5-4 Hz) and broadband gamma activity (55-145 Hz) was calculated. As illustrated in FIGS. 3A and 3B, Tau Modulation Curves were calculated as disclosed herein in steps of 0.2 s across each 2.56 s-long observation window obtained over the Propofol-induced LOC. The signal-to-noise ratio (SNR) across each observation window was calculated as a measure of the strength of the modulation between the slow wave oscillations and the broadband gamma activity.

The slow wave activity exhibited two distinct stages as it traveled through the entire hemisphere throughout the Propofol-induced anesthesia of the monkeys (FIG. 3C). The first stage (STAGE-I) encompassed an approximately one-minute-long period following the Propofol injection. The spatial dynamics of this period resembled those of previously reported default networks, which are known to have vast anatomical connections through the brain, and are considered to relate to higher-order functions. The second stage (STAGE-II) encompassed approximately 5-20 minutes after Propofol injection. In this period, the spatiotemporal dynamics of the slow-wave activity exhibited the propagation of the Propofol's effect on individual brain regions. In this, the slow-wave preferentially originated from the lateral sulci and anterior cingulate gyrus and propagated along the anterior-posterior direction.

The results of these experiments shed some light on the propagation of slow-wave activity throughout the cortex. This understanding could lead to new diagnostic methods that characterize pathologies directly from the brain's slow-wave activity.

### Example 3: Tracking Mood & Attention

Network interactions are associated with specific frequency rhythms. Additionally, these rhythmically encoded networks can become distorted in various psychiatric and neurologic diseases. The method could be useful in potentially measuring changes in mood and other neurologic conditions.

### Example 4: Tau-Modulation Curve Comparison for Different Cognitive Tasks

To compare Tau-Modulation Curve characteristics based on electrocorticographic signals obtained during different cognitive and behavioral tasks, the following experiments were conducted.

Electrocorticographic signals were recorded from one human subject who underwent temporary placement of electrode grids over his right hemisphere prior to surgical resection of epileptogenic foci. The subject was implanted with several subdural Electrocorticographic (ECoG) grids that covered the frontal, parietal and temporal lobes, as illustrated on the brain diagram in FIG. 10. The ECoG grids included platinum-iridium electrodes in various configurations with an exposed diameter of 3 mm and an inter-electrode spacing of 6-10 mm (PMT Corporation, Chanhassen, Minn.). ECoG grid coverage and implant duration were dictated solely by the clinical requirements without considering the research study.

Following the implantation, patient-specific brain models were generated based on pre-operative structural magnetic resonance images (MRI) using the FreeSurfer software. The pre-operative MRI was co-registered with a postoperative computed tomography (CT) scan to localize electrode locations on the patient-specific brain models. The NeuralAct toolbox and MATLAB 2015a (The MathWorks, Inc., Natick, Mass., United States) were used to generate brain plots.

Recording was accomplished at the patient's bedside using the general-purpose BCI2000 software, which interfaced with a g.Hlamp biosignal acquisition device (g.tec, Graz, Austria). A splitter box routed signals simultaneously to the clinical monitoring system and to the BCI2000/amplifier system, and thereby supported continuous clinical monitoring. The signals were amplified, digitized at 1200 Hz, and stored by the BCI2000.

During the recordings, the subject participated in five behavioral tasks: solving Rubik's cubes (T1), tickling chin (T2), sticking out tongue (T3), pursing lips (T4), and listening (T5). Tasks T1, T3, and T4 required active subject participation; and T2 and T5 were administered to the subject. Text presented on a computer screen cued the subject for each task. The tasks were presented in sequence, executed for 15 seconds for tasks T1-T4 and 17-36 seconds for T5, and interleaved with rest periods (T6) that lasted for 15 seconds. The experiment consisted of 5 repetitions of this sequence over the course of approximately 10 minutes.

Each set of recorded ECoG signals for each task was processed to produce Tau-Modulation Curves, as disclosed herein. To pre-process the data, the recorded signals were visually inspected and any signals associated with non-functional electrodes were rejected. A high-pass finite impulse response (FIR) filter at 0.5 Hz was used to remove slow drifts, a common average reference spatial filter was used to remove common noise across all channels, and two notch FIR filters at 58-62 Hz and 118-122 Hz were used to remove line noise and its first harmonic.

The pre-processed ECoG signals were transformed into Tau-Modulation curves using the methods disclosed herein. Wideband low-frequency (WBLF) signals and broadband gamma envelope signals were extracted from the raw ECoG signals (FIG. 1A). An FIR lowpass filter (<30 Hz) was used to obtain the wideband low-frequency (WBLF) signal. The wideband characteristics of this filter preserved the shape and amplitude of the neuronal oscillation. An FIR bandpass filter followed by a Hilbert transform was used to extract the broadband gamma envelope (70-170 Hz). The resulting WBLF signal and broadband gamma envelope were downsampled from the original 1200 Hz to 200 Hz.

The wideband low-frequency signal was normalized to remove the 1/f characteristic (FIG. 1B). A Hamming window followed by a fast Fourier transform (FFT) was used to extract the signal's amplitude and phase spectrum. A least square regression was performed to determine a linear fit of the amplitude spectrum within the 1-30 Hz log-log spaced range. This linear fit was used to normalize the amplitude spectrum while keeping the phase spectrum constant. An inverse FFT was performed on this normalized amplitude spectrum to transform the frequency-domain signal back into the time-domain to produce the normalized-WBLF signal (FIG. 1B).

A cross-correlation between the normalized-WBLF and the broadband gamma envelope was performed to produce the Tau-Modulation Curve (TMC) as illustrated in FIG. 1C. The TMCs were calculated using 1.28 s-long observation windows that were moved in steps of 0.04 s. The strength and frequency of WBLF coupling with broadband gamma activity were determined based on further analysis of the TMCs.

To determine the strength of WBLF coupling with broadband gamma activity, a signal-to-noise ratio (SNR) for each task condition was calculated as the ratio between the variance across all TMC samples, and the average variance of each individual TMC sample, as illustrated in FIG. 1D. SNR>1 indicates that WBLF is modulating broadband gamma activity and SNR=1 indicates no modulation. Thus, the SNR values serve as an indication of the strength of phase-amplitude coupling (PAC) between WBLF and broadband gamma activity.

To determine the frequency of the WBLF coupling with the broadband gamma activity, the average TMC for each task condition was calculated and a matching pursuit filter was applied to determine the fundamental oscillation frequency of the average TMC as illustrated in FIG. 1E. Matching pursuit is a technique that iteratively approximates the original signal with a linear combination of 1-dimensional Gabor functions to determine the frequency of PAC between the WBLF and broadband gamma activity (FIG. 1E).

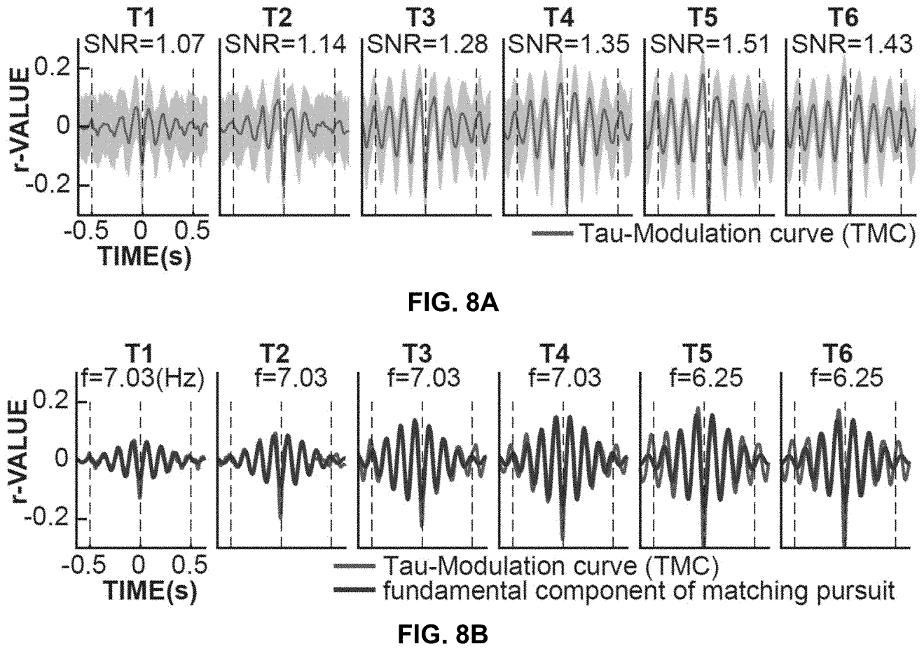

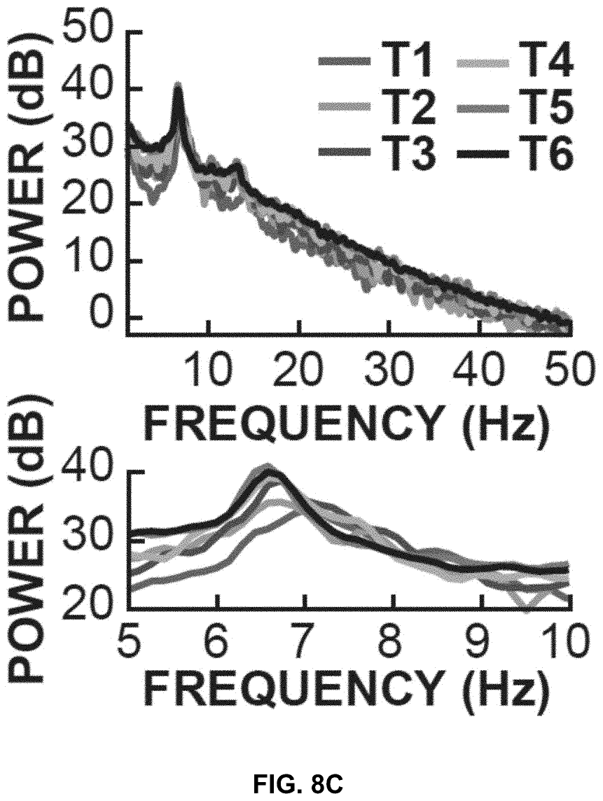

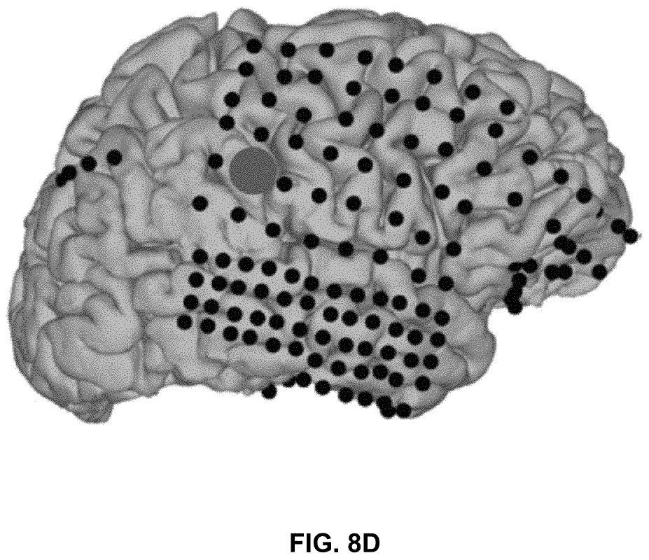

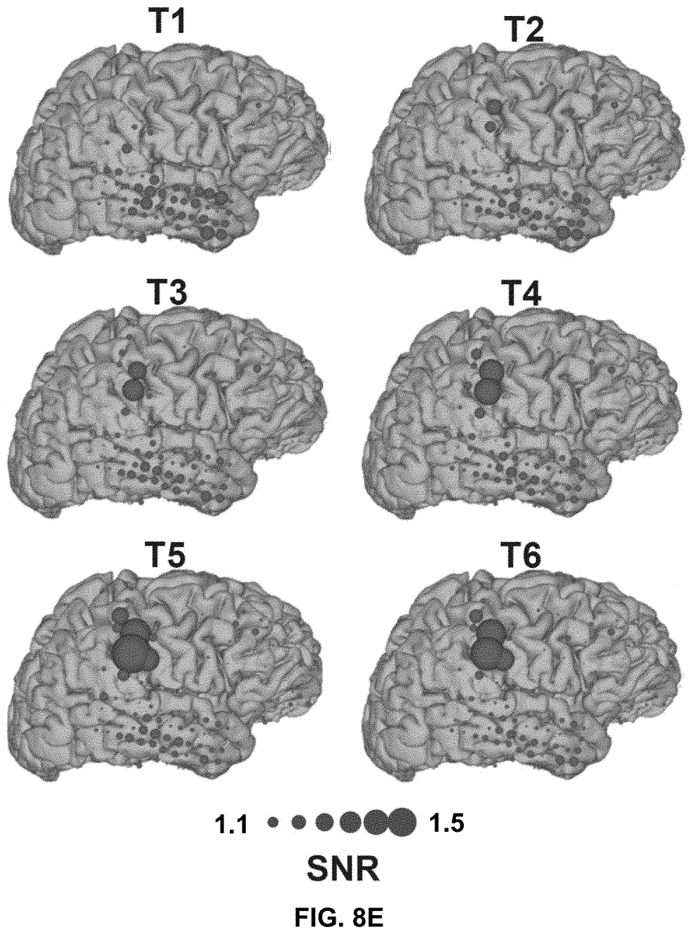

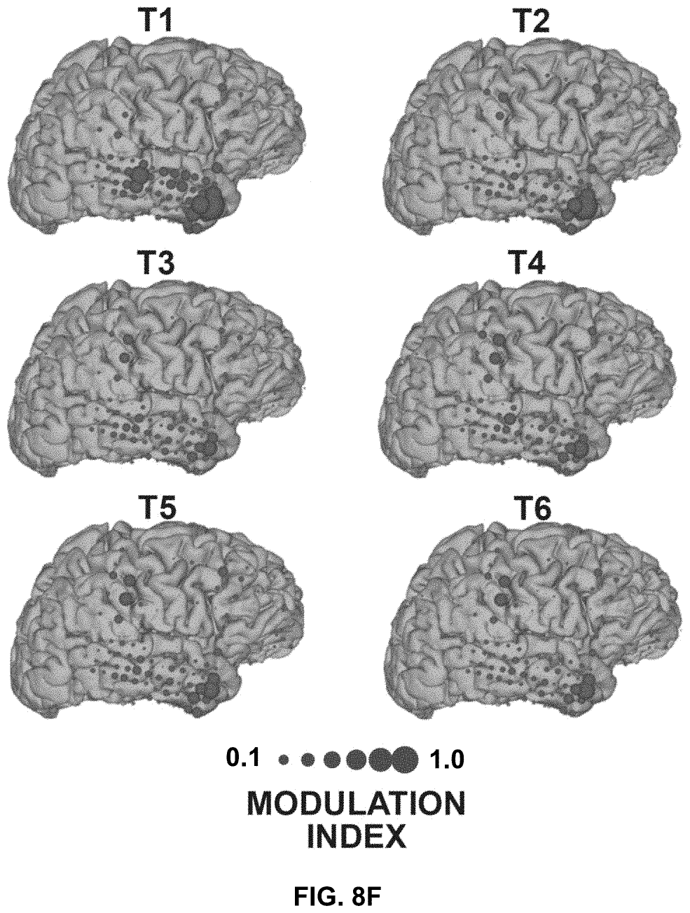

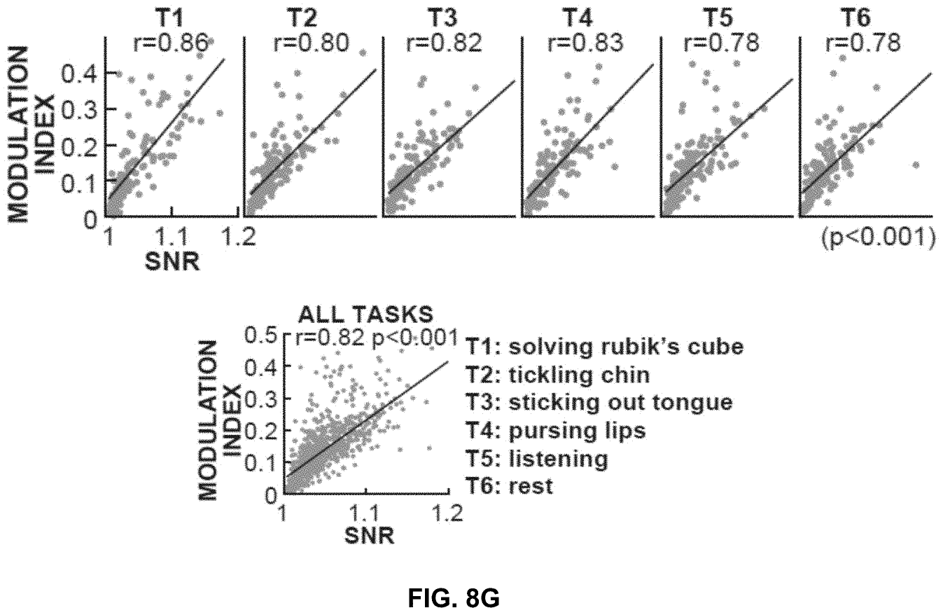

FIG. 8A is a series of graphs summarizing the TMC strengths obtained as described above for each of the six cognitive and behavioral tasks. FIG. 8E is a series of brain maps summarizing the spatial distribution of SNRs obtained for each task. FIGS. 8A and 8E demonstrate that the strength of the Tau-Modulation Curve during different behavioral and cognitive tasks engages different cortical networks. For example, when the subject is “Solving Rubik's Cube”, the task-irrelevant temporal lobe exhibits higher modulation strength than the task-relevant regions surrounding the central sulcus. However, when the subject is “Listening”, the Tau-Modulation strength reverses, i.e., the regions around the now task-irrelevant central sulcus exhibits higher modulation strength than the now task-relevant regions within the temporal lobe. These results indicate that the Tau-Modulation strength might serve as a useful index to identify task-relevant neuronal networks.

To verify these results, a control analysis was performed by calculating the modulation index (MI) between the low-frequency oscillation (5-9 Hz) and broadband gamma envelope (70-170 Hz) using previous existing methods. The MIs for each task were correlated with the corresponding SNRs produced using the same ECoG measurements. FIG. 8G is a series of graphs summarizing the Spearman correlations between the MIs and SRs for each task and for all tasks combined. As shown in FIG. 8G, the strength of Tau-Modulation (SNR) is highly correlated with the corresponding modulation index (MI) (r=0.82, p<0.001, Spearman correlation). This comparison confirmed that the TMC analysis yielded similar results to those of previous studies that investigated PAC in ECoG signals.

FIG. 8B contains a series of graphs summarizing the fundamental frequency of the Tau-Modulation Curves for the different behavioral and cognitive tasks. The fundamental frequency varied across tasks to accommodate the varying behavioral and cognitive demands.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

What is claimed is:

1. A computer-implemented method for tracking a brain state and mapping a functional brain organization of a subject, the method comprising:
a. receiving, at a computing device, a plurality of brain activity measurements indicative of brain activity of the subject;
b. extracting, using the computing device, a plurality of wideband low frequency (WBLF) signals from the plurality of brain activity measurements;
c. extracting, using the computing device, a plurality of broadband gamma envelope signals from the plurality of brain activity measurements;
d. calculating, using the computing device, a cross-correlation between the plurality of wideband low frequency (WBLF) signals and the plurality of broadband gamma envelope signals to produce at least one Tau Modulation Curve; and
e. displaying, using the computing device, the at least one TMC, wherein the at least one TMC is indicative of the WBLF modulation of broadband gamma activity in the brain of the subject.

2. The method of claim 1, wherein the plurality of brain activity measurements comprises at least one of electroencephalographic (EEG) signals, magnetoencephalographic (MEG) signals, electrocorticographic (ECoG) signals, stereo electroencephalography (SEEG) signals, functional magnetic resonance (fMRI) signals, and functional near-infrared optical imaging (fNRI) signals.

3. The method of claim 1, wherein extracting the plurality of WBLF signals comprises applying, using the computing device, an FIR lowpass filter (<30 Hz) to the plurality of brain activity measurements.

4. The method of claim 3, further comprising normalizing, using the computing device, the plurality of WBLF signals to remove the effect of 1/f power law scaling.

5. The method of claim 4, wherein normalizing the plurality of WBLF signals comprises:
a. applying, using the computing device, a Hamming window and a fast Fourier transform to the plurality of WBLF signals to obtain a WBLF amplitude spectrum and a WBLF phase spectrum;
b. obtaining, using the computing device, a least-squares linear regression fit of the WBLF amplitude spectrum over a 1-30 Hz log-log spaced range;
c. normalizing, using the computing device, the WBLF amplitude spectrum using the least-squares linear regression fit to obtain a normalized amplitude spectrum; and
d. performing, using the computing device, an inverse fast Fourier transform to the normalized amplitude spectrum to obtain a plurality of normalized WBLF signals.

6. The method of claim 1, wherein extracting the plurality of broadband gamma envelope signals comprises applying, using the computing device, an FIR bandpass filter (70-170 Hz) and a Hilbert transform to the plurality of brain activity measurements.

7. The method of claim 1, further comprising calculating, using the computing device, a TMC-strength for each of the at least one TMCs, each TMC-strength indicative of a strength of the WBLF modulation of broadband gamma activity in the brain of the subject, wherein each TMC-strength comprises a signal-to-noise ratio (SNR) for each of the at least one TMCs, each SNR comprising a ratio of an average variance of each TMC and an average variance of all of the at least one TMCs.

8. The method of claim 7, wherein a TMC-strength value of at least 1 is indicative of a presence of WBLF modulation of broadband gamma activity in the brain of the subject.

9. The method of claim 8, further comprising calculating, using the computing device, a TMC-frequency for each of the at least one TMCs, each TMC-frequency indicative of a frequency of WBLF modulation of broadband gamma activity in the brain of the subject, wherein calculating the TMC-frequency comprises:
a. calculating, using the computing device, an average TMC for each of the at least one TMCs; and
b. applying, using the computing device, a matching pursuit filter to determine a fundamental oscillation frequency of each average TMC, wherein the fundamental oscillation frequency is the TMC-frequency.

10. The method of claim 9, wherein displaying the at least one Tau Modulation Curve (TMC) further comprises displaying, using the computing device, a TMC-strength map and a TMC-frequency map, the TMC-strength and TMC-frequency maps comprising a plurality of TMC-strengths and a plurality of TMC-frequencies mapped to a corresponding plurality of brain positions at which the subset of brain activity measurements used to produce each TMC was obtained, respectively.

11. A system for tracking a brain state and mapping a functional brain organization of a subject, the system comprising a computing device, the computing device comprising at least one processor, the at least one processor configured to:
a. receive a plurality of brain activity measurements indicative of brain activity of the subject;
b. extract a plurality of wideband low frequency (WBLF) signals from the plurality of brain activity measurements;
c. extract a plurality of broadband gamma envelope signals from the plurality of brain activity measurements;
d. calculate a cross-correlation between the plurality of wideband low frequency (WBLF) signals and the plurality of broadband gamma envelope signals to produce at least one Tau Modulation Curve (TMC); and
e. display the at least TMC, wherein the at least one TMC is indicative of the WBLF modulation of broadband gamma activity in the brain of the subject.

12. The system of claim 11, wherein the plurality of brain activity measurements comprises at least one of electroencephalographic (EEG) signals, magnetoencephalographic (MEG) signals, electrocorticographic (ECoG) signals, stereo electroencephalography (SEEG) signals, functional magnetic resonance (fMRI) signals, and functional near-infrared optical imaging (fNRI) signals.

13. The system of claim 11, wherein the at least one processor is further configured to extract the plurality of WBLF signals by applying an FIR lowpass filter (<30 Hz) to the plurality of signals indicative of brain activity.

14. The system claim 13, wherein the at least one processor is further configured to normalize the plurality of WBLF signals to remove the effect of 1/f power law scaling.

15. The system of claim 14, wherein the at least one processor is further configured to normalize the plurality of WBLF signals by:
a. applying a Hamming window and a fast Fourier transform to the plurality of WBLF signals to obtain a WBLF amplitude spectrum and a WBLF phase spectrum;
b. obtaining a least-squares linear regression fit of amplitude spectrum with a 1-30 Hz log-log spaced range;
c. normalizing the amplitude spectrum using the least-squares linear regression fit to obtain a normalized amplitude spectrum; and
d. performing an inverse fast Fourier transform to the normalized amplitude spectrum to obtain a plurality of normalized WBLF signals.

16. The system of claim 11, wherein the at least one processor is further configured to extract the plurality of broadband gamma envelope signals by applying an FIR bandpass filter (70-170 Hz) and a Hilbert transform to the plurality of brain activity measurements.

17. The system of claim 11, wherein the at least one processor is further configured to calculate a TMC-strength for each of the at least one TMCs, wherein:
a. each TMC-strength comprises a signal-to-noise ratio (SNR) for each of the at least one TMCs, each SNR comprising a ratio of an average variance of each TMC and an average variance of all of the at least one TMCs; and
b. a TMC-strength value of at least 1 is indicative of a presence of WBLF modulation of broadband gamma activity in the brain of the subject.

18. The system claim 17, wherein the at least one processor is further configured to calculate a TMC-frequency for each of the at least one TMCs, each TMC-frequency indicative of a frequency of WBLF modulation of broadband gamma activity in the brain of the subject, wherein calculating the TMC-frequency comprises:
a. calculating an average TMC for each of the at least one TMCs; and
b. applying a matching pursuit filter to determine a fundamental oscillation frequency of each average TMC, wherein the fundamental oscillation frequency is the TMC-frequency.

19. The system of claim 18, wherein the at least one processor is further configured to display at least one of a TMC-strength map and a TMC-frequency map, the TMC-strength and TMC-frequency maps comprising a plurality of TMC-strengths and a plurality of TMC-frequencies mapped to a corresponding plurality of brain positions at which the subset of brain activity measurements used to produce each TMC was obtained, respectively.

20. The system of claim 11, further comprising a brain activity monitoring device operatively coupled to the computing device, the brain activity monitoring device configured to obtain the plurality of brain activity measurements, the brain activity monitoring device comprising one of an electroencephalographic system, a magnetoencephalographic (MEG) system, an electrocorticographic (ECoG) system, a stereo electroencephalography (SEEG) system, a functional magnetic resonance (fMRI) system, and a functional near-infrared optical imaging (fNRI) system.
